(19) United States
(12) Patent Application Publication
(10) Pub. No.: US 2022/0313913 A1
(43) Pub. Date: Oct. 6, 2022

(54) Method and Apparatus for Regulating and Monitoring Discharge from a Syringe or Other Fluid Dispensing Apparatus

(71) Applicant: SAFEPUSH, LLC, Marrero, LA (US)

(72) Inventor: Kenneth P. Perry, Youngsville, LA (US)

(21) Appl. No.: 17/714,082

(22) Filed: Apr. 5, 2022

Related U.S. Application Data
(60) Provisional application No. 63/171,149, filed on Apr. 6, 2021.

(51) Int. Cl. A61M 5/19; A61M 5/20

(52) U.S. Cl. CPC A61M 5/19; A61M 5/2053; A61M 2005/3128

(57) ABSTRACT

A method and apparatus for controlling and regulating fluid output from a syringe or other dispensing device that is not dependent on use of a specialized technique by a user. A fluid chamber can volumetrically increase or decrease depending upon the pressure of fluid being displaced from the chamber. The fluid chamber defines an internal volume that will automatically and dynamically adjust in order to maintain a desired output fluid flow rate from an outlet that will not exceed a predetermined maximum flow rate. Little or no frictional forces are imparted. The adjustable fluid chamber can exhibit a resistance to expansion that corresponds to a desired output fluid flow rate, while maintaining a substantially constant fluid pressure required to create a desired output flow rate through an outlet.

### BACKGROUND OF THE PRESENT INVENTION

### 1. Field of the Invention

The present invention pertains to a method and apparatus for controlling a flow rate of fluid (such as, for example, liquid medication) dispensed via a syringe or other dispensing apparatus, including, without limitation, into an intravenous (IV) line port or hypodermic needle. More particularly, the present invention pertains to a method and apparatus for regulating a dispensing rate of said fluid (such as, for example, liquid medication) wherein said dispensing rate does not exceed a predetermined maximum flow rate.

### 2. Description of Related Art

It is frequently beneficial in many different industries and applications to control or regulate fluid flow rate in order to achieve desired results and/or to prevent possible adverse outcomes. One such application is the medical treatment of patients. Although specific situations can vary, one particular medical application wherein fluid flow rate must be carefully regulated is the administration of drugs and other liquid medicaments to patients.

Currently, drugs, medicaments and other fluids are commonly administered to patients in a healthcare setting. Some drugs, including those classified as “high alert” and critical care medications, can cause adverse reactions in patients, detrimental effects and even death if administered at a rate above the manufacturer's suggested flow rate. During emergency events and, in particular, when critical care medications are being administered, injury or death may occur to a patient if medications are administered at a greater rate than what is prescribed. Conventional devices to regulate dispensing rates exist in various modalities; however, such conventional devices are typically large, expensive and complex. In many cases, said conventional devices require advance programming that can be difficult and time-consuming to accomplish. Such conventional devices frequently malfunction and, further, are generally not suitable to operate in the fast-paced environment of emergency rooms and other healthcare facilities.

Because a simple-to-use, portable, relatively inexpensive, and readily accessible device for predictably regulating the flow rate of critical care medication dispensed to a patient is currently not available, clinicians typically administer such medication while watching a clock or wristwatch in order to estimate flow rate of outlet discharged from a syringe. This method is subject to inaccuracy and frequently requires a doctor or healthcare professional to direct his or her attention away from a patient in order to simultaneously monitor the dispensing flow from the syringe and the time passing on a wristwatch or clock.

Additionally, patient vital signs should be monitored while administering medication in order to prevent detrimental effects and to monitor beneficial results on the patient. The aforesaid process of attempting to simultaneously monitor patient vital signs, a watch or clock, and the dispensing fluid flow from a syringe creates a high probability for errors in flow rate estimation, while also increasing likelihood that signs or symptoms exhibited by a patient will be missed or overlooked. It is known that such errors, which can frequently prove to be deadly, often result from administering medication to patients at flow rates that are higher than desired (such as, for example, rates that are greater than a manufacturer's predetermined maximum administration rate).

Conventional syringe pumps currently used in the health care industry are programmable devices that can receive a syringe and can be programmed for a desired output fluid flow rate from said syringe. The syringe pump will automatically pump the medicine from the syringe at said desired fluid flow rate. However, it is known that said conventional syringe pumps periodically malfunction and dispense medication at higher flow rate(s) than the programmed flow rate.

Thus, there is a need for a reliable, effective, inexpensive and user-friendly means for controlled fluid flow rate regulation from a syringe or other dispensing apparatus, particularly (but not exclusively) during administration of fluid medicaments. A clinician or other user should be able to beneficially avoid delay and safely administer medicine at or below a desired maximum fluid flow rate, while simultaneously monitoring a patient receiving such medicine. Further, said means for controlled fluid flow rate regulation should be compatible for use with a conventional syringe, as well as a syringe pump, such that a desired output flow rate will be maintained even if said conventional syringe pump malfunctions.

### SUMMARY OF THE INVENTION

The present invention comprises a method and apparatus for controlling and regulating fluid output from a syringe or other dispensing apparatus that is not dependent on use of a specialized technique by a user or operator. The present invention regulates fluid output flow rate by employing a primary fluid chamber that is capable of automatically expanding (and/or contracting) volumetrically based on flow rate and corresponding pressure of fluid being displaced from said primary fluid chamber.

Importantly, the present invention does not require a user to divert fluid or medication out of a primary fluid chamber, and a user maintains constant and direct access to said fluid or medication at all relevant times. Put another way, all fluid or medication that has not been displaced through an outlet remains available for dispensing without requiring a user to perform a secondary procedure or operation.

In a preferred embodiment, the present invention comprises an apparatus defining a primary fluid chamber having an outlet and a means for displacing fluid from said primary fluid chamber through said outlet. As fluid in said primary fluid chamber is acted upon by an external force to pump said fluid from said chamber (such as, for example, a plunger being pushed into a barrel of a syringe), said fluid becomes pressurized and is displaced through said outlet. As the flow rate of fluid displaced through said outlet increases, the corresponding pressure of fluid in said primary fluid chamber also increases.

If the flow rate of said fluid flowing through said outlet exceeds a certain predetermined flow rate (that is, a predetermined maximum allowable flow rate), the internal volume of said primary fluid chamber can automatically expand in order to reduce the pressure of said fluid in said primary fluid chamber. According to Poiseuille's Law and principles of fluid mechanics, by reducing said fluid pressure in said primary fluid chamber to a known pressure, a corresponding known or predictable output fluid flow rate can be maintained.

Generally, the method of the present invention can be employed in devices of virtually any size or volume, while accommodating virtually any flow rate. Expansion and contraction of said primary fluid chamber volume is accomplished with little or no frictional forces so as not to impart additional or unpredictable fluid pressure or forces into the system. The primary fluid chamber of the present invention should beneficially exhibit a resistance to expansion that corresponds to a desired output fluid flow rate.

The present invention can comprise a specialty syringe having a barrel defining a primary fluid chamber having an internal volume necessary to contain an initial volume of fluid or medication, as well as a plunger movably disposed in said barrel. A supplemental chamber having a secondary volume can be disposed within said plunger or otherwise in proximity to said syringe barrel. A movable membrane or other dividing structure forms a boundary between volumes of said primary and supplemental chambers. A biasing member provides biasing force to said membrane or other dividing structure.

Said biasing force acting on said membrane or dividing structure should ideally comprise a substantially constant force. Said constant force can be created with a constant force compression spring, an elastomer, a bellow-shaped structure, regulated pneumatics, or any number of other devices and/or mechanisms that can generate constant force in order to maintain a desired substantially constant fluid pressure.

In an alternative embodiment, the present invention can comprise an apparatus that can be attached to the output port of a conventional syringe, or in a flow line or conduit wherein a substantially constant fluid flow rate is desired. Said alternative embodiment similarly regulates fluid pressure to maintain a desired flow rate without exceeding a predetermined maximum fluid flow rate, but also allows a user to employ a conventional syringe. Said alternative embodiment apparatus for controlling and regulating fluid output can be attached to the outlet of a conventional syringe or disposed in-line within any other fluid conduit.

In another alternative embodiment, the present apparatus senses fluid flowrate by correlating a pressure differential measured across hydraulic element(s) to a fluid flow rate; said hydraulic element(s) can include, without limitation, check valve(s), orifice(s), membrane(s), or combinations thereof, and said output fluid flow rate can be determined through calculations and/or empirical testing. Said alternative embodiment can also provide an audible or visual notification if said fluid flow rate exceeds a desired flow rate.

The present invention can be programmed in advance for virtually any desired fluid flow rate, and can beneficially monitor, record and/or display desired data (including, without limitation, date, time, duration of administration, flow rate and volume of medication administered). Further, said alternative embodiment can also transmit data regarding output fluid flow rate, via wired or wireless connection, to monitors and/or other display devices in order to display flow rate in real time. Said data can also be collected and stored for later retrieval and/or use.

Among other uses, data from the present invention can aid in validation of fluid flow rate (for example, for malpractice issues), can be incorporated into patient charts and can be used by health care providers for monetization and validation of procedure(s) performed. Such data can also be extracted from the present invention using conventional medical scanners or using other known methods of data download.

### BRIEF DESCRIPTION OF THE DRAWINGS

The foregoing summary, as well as any detailed description of the preferred embodiments, is better understood when read in conjunction with the drawings and figures contained herein. For the purpose of illustrating the invention, the drawings and figures show certain preferred embodiments. It is understood, however, that the invention is not limited to the specific methods and devices disclosed in such drawings or figures.

FIG. 1 depicts a side sectional view of a first embodiment output flow regulating apparatus of the present invention.

FIG. 2 depicts a side sectional view of a first embodiment output flow regulating apparatus of the present invention in a first position.

FIG. 3 depicts a side sectional view of a first embodiment output flow regulating apparatus of the present invention in a second position.

FIG. 4 depicts a side sectional view of a first embodiment output flow regulating apparatus of the present invention in a third position.

FIG. 5 depicts a side sectional view of a first embodiment output flow regulating apparatus of the present invention in a fourth position.

FIG. 6 depicts a side sectional view of a first embodiment output flow regulating apparatus of the present invention in fifth position.

FIG. 7 depicts a side sectional view of a first embodiment output flow regulating apparatus of the present invention in said first position.

FIG. 8 depicts a perspective and partially cut-away view of a second embodiment output flow regulating apparatus of the present invention in a first position.

FIG. 9 depicts a perspective and partially cut-away view of a second embodiment output flow regulating apparatus of the present invention.

FIG. 10 depicts a side sectional view of a second embodiment output flow regulating apparatus of the present invention.

FIG. 11 depicts a perspective view of a third embodiment output flow regulating apparatus of the present invention.

FIG. 12 depicts a side sectional view of a third embodiment output flow regulating apparatus of the present invention.

FIG. 13 depicts a side sectional view of a third embodiment output flow regulating apparatus of the present invention installed in connection with a conventional syringe.

FIG. 14 depicts a side perspective view of a third embodiment output flow regulating apparatus of the present invention installed in connection with a conventional syringe and a flow conduit.

FIG. 15 depicts a side perspective view of a third embodiment output flow regulating apparatus of the present invention installed in connection with a conventional syringe equipped with a hypodermic needle.

FIG. 16 depicts a perspective view of an orifice attachment of the present invention.

FIG. 17 depicts a side sectional view of an orifice attachment of the present invention.

FIG. 18 depicts a side perspective view of an orifice attachment of the present invention installed on a syringe.

FIG. 19 depicts a side sectional view of an orifice attachment of the present invention installed on an output flow regulating apparatus of the present invention.

FIG. 20 depicts a side perspective and partially exploded view of a syringe having an adjustable orifice disk.

FIG. 21 depicts a side perspective view of a syringe having an adjustable orifice disk.

FIG. 22 depicts a side sectional view of a first alternative embodiment plunger apparatus of the present invention.

FIG. 22A depicts a perspective sectional view of said first alternative embodiment plunger apparatus of the present invention.

FIG. 23 depicts a side sectional view of a second alternative embodiment plunger apparatus of the present invention.

FIG. 23A depicts a perspective sectional view of said second alternative embodiment output flow regulating apparatus of the present invention.

FIG. 24 depicts a detailed sectional view of an output flow regulating apparatus of the present invention further comprising a check valve assembly.

FIG. 25 depicts a detailed perspective view of an adjustable-rate cap assembly of the present invention.

FIG. 26 depicts a side sectional view of an output flow measurement apparatus of the present invention.

FIG. 27 depicts a side perspective view of an output flow measurement apparatus of the present invention equipped with output flow regulating apparatus having a supplemental fluid container.

FIG. 28 depicts a side perspective view of an output flow measurement apparatus of the present invention installed on an output flow regulating apparatus of the present invention.

FIG. 29 depicts a side sectional view of an output flow regulating apparatus equipped with output flow measurement apparatus of the present invention.

FIG. 30 depicts a detailed view of highlighted area “A” depicted in FIG. 29.

FIG. 31 depicts a side sectional and partially exploded view of a plunger stop mechanism of the present invention.

FIG. 32 depicts a side sectional view of a plunger stop mechanism of the present invention.

FIG. 33 depicts a side sectional view of an alternative plunger stop mechanism of the present invention.

FIG. 34 depicts a perspective and partially exploded view of a said alternative plunger stop mechanism of the present invention.

### DETAILED DESCRIPTION OF A PREFERRED EMBODIMENT

The present invention comprises a method and apparatus for controlling and regulating fluid output from a dispensing device such as, for example, a syringe or other container, that is not dependent upon use of a specialized technique by an operator. By way of illustration, but not limitation, a preferred flow rate for certain critical care drugs can differ depending upon the manufacturer's recommended flow rate and drug concentrations (e.g., 1 cc/min, 2 cc/min, 5 cc/min or some other flow rate). Thus, users of the present invention can include, but are not necessarily limited to, emergency and trauma personnel, paramedics and veterinarians.

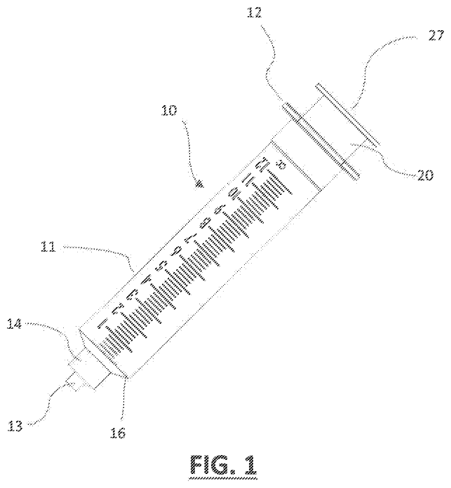

Referring to the drawings, FIG. 1 depicts a side view of a first embodiment output flow regulating apparatus 10 of the present invention in a first position. As depicted in FIG. 1, output flow regulating apparatus 10 generally resembles a conventional syringe having a barrel 11 defining an inner chamber, with distal end 16 at one end of said barrel 11, and barrel or finger flanges 12 at the opposite end of barrel 11. Said barrel 11 further comprises fluid outlet 13 and connection assembly 14. Although other connection assemblies can be optionally employed, in a preferred embodiment said connection assembly 14 comprises a Luer lock connection that is well known to those having skill in the art. Said connection assembly 14 can permit operational attachment of said barrel 11 (and fluid outlet 13) to another device or component such as, for example, an output tube or hypodermic needle. Output flow regulating apparatus 10 further comprises a movable plunger 20 that is slidably received within the inner chamber of barrel 11; plunger 20 has thumb flange 27 at one end.

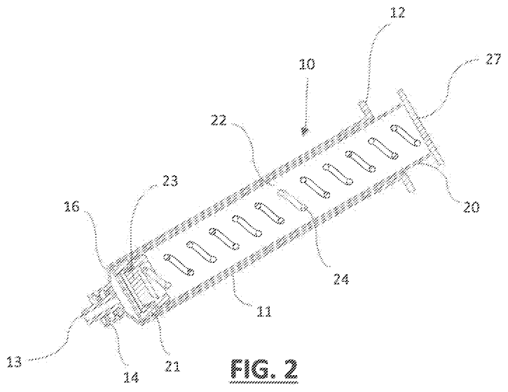

FIG. 2 depicts a side sectional view of a first embodiment output flow regulating apparatus 10 of the present invention in a first position. Output flow regulating apparatus 10 comprises barrel 11 defining an inner chamber. Barrel 11 has distal end 16 at one end of said barrel 11, and barrel or finger flanges 12 at the opposite end of barrel 11. Said barrel 11 further comprises fluid outlet 13 and connection assembly 14.

Still referring to FIG. 2, first embodiment output flow regulating apparatus 10 further comprises movable plunger 20 that is slidably received within the inner chamber defined by barrel 11. Said moveable plunger 20 further comprises plunger piston 21 at one end, and plunger or thumb flange 27 at its opposite end. Plunger piston 21 can be beneficially constructed of an elastomer, plastic or other material that can provide a dynamic fluid seal—or at least fit tightly—against the inner surface of barrel 11. It is to be observed that barrel 11 can be virtually any size, while said inner chamber defined by said barrel 11 can be dimensioned to accommodate virtually any desired volume of liquid. Further, said barrel 11 can be constructed of virtually any material; in the preferred embodiment, said barrel 11 can be constructed of plastic or glass.

In the position depicted in FIG. 2, plunger 20 is shown as being substantially fully inserted or pushed within said inner chamber defined by barrel 11, such that plunger piston 21 is proximate to or in contact with distal end 16 of barrel 11. As depicted in FIG. 2, plunger 20 includes a supplemental or secondary inner chamber 22 disposed within said plunger 20. Plunger inner piston 23 is movably disposed within said inner chamber 22; force is applied to plunger inner piston 23 to bias said plunger inner piston 23 in a direction away from plunger flange 27 and toward outlet 13. In a preferred embodiment, said biasing force can be applied by compression spring 24, while the volume of secondary inner chamber 22 should beneficially be substantially the same or larger than the volume of the inner chamber defined by barrel 11. In the position depicted in FIG. 2, output flow regulating apparatus 10 can be used to draw fluid from a vial or other container into said inner chamber defined by barrel 11 for later dispensing in much the same manner as a conventional syringe.

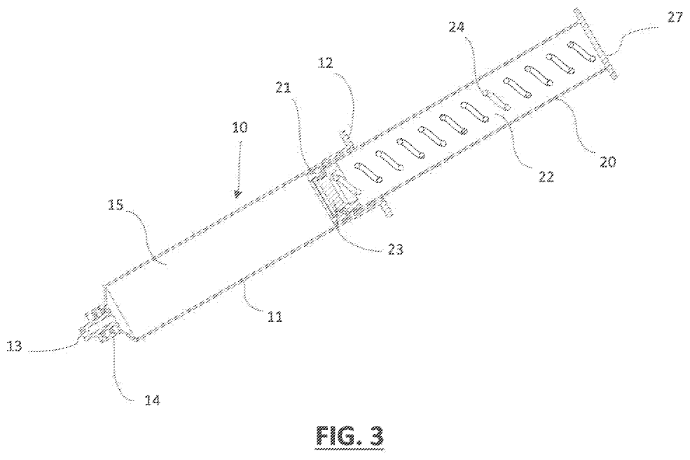

FIG. 3 depicts a side sectional view of said first embodiment output flow regulating apparatus 10 of the present invention depicted in FIG. 2, but with plunger 20 in a second position. In this position, plunger 20 is substantially fully withdrawn or pulled from the inner chamber 15 defined by barrel 11. Supplemental or secondary inner chamber 22 is disposed within said plunger 20, while plunger inner piston 23 is fully seated within plunger piston 21, and biasing force is applied to said plunger inner piston 23 by compression spring 24. In the position depicted in FIG. 3, it is to be understood that fluid (such as medicine) has been drawn from a vial or other container into primary inner chamber 15 of barrel 11 for dispensing through outlet 13.

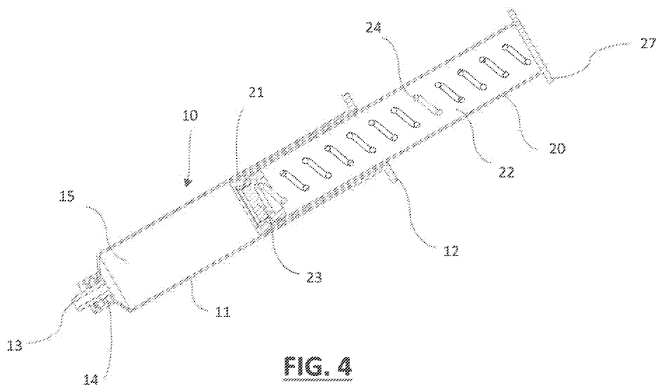

FIG. 4 depicts a side sectional view of first embodiment output flow regulating apparatus 10 of the present invention depicted in FIG. 3, but with plunger 20 in a third position. In this position, plunger 20 is depicted as being partially pushed into primary inner chamber 15 of barrel. Supplemental or secondary inner chamber 22 is contained within said plunger 20, while plunger inner piston 23 remains fully seated in plunger piston 21. Biasing force is applied to said inner plunger piston 23 by compression spring 24. Plunger piston 21 is beneficially constructed of an elastomer, plastic or other material that provides a dynamic fluid seal (or at least a tight fit) against the inner surface of primary inner chamber 15 of barrel 11, and acts to pump fluid from said primary inner chamber 15. In the position depicted in FIG. 4, it is to be understood that some volume of previously-loaded fluid has been displaced from primary chamber 15 of barrel 11 by plunger 20 via fluid outlet 13. However, the flow rate of fluid flowing through fluid outlet 13, and the corresponding fluid pressure in primary inner chamber 15 of barrel 11, have not been exceeded (as reflected by the fact that plunger inner piston 23 remains fully seated in plunger piston 21).

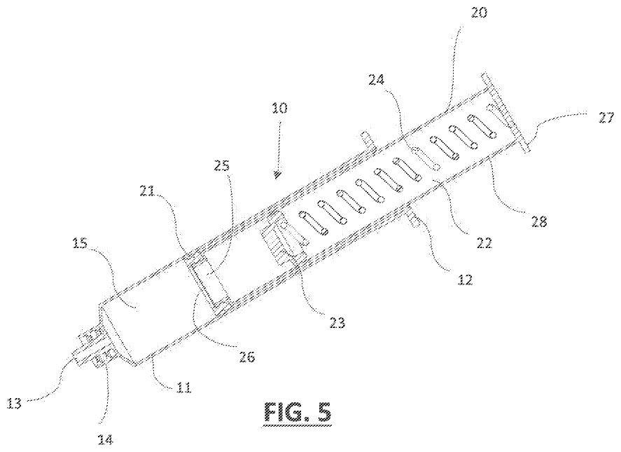

FIG. 5 depicts a side sectional view of a first embodiment output flow regulating apparatus 10 of the present invention depicted in FIG. 4, but with plunger 20 depicted in a fourth position. In this position, plunger 20 is depicted as being partially pushed into said primary inner chamber 15 of barrel 11. As with conventional syringes, as plunger 20 is pushed deeper into primary inner chamber 15 of barrel 11, the volume of said primary inner chamber 15 is effectively reduced between plunger piston 21 and outlet 13, thereby causing liquid or other fluid to be pumped or displaced from said primary inner chamber 15 through said outlet 13.

Because outlet 13 has a relatively small diameter relative to primary inner chamber of 15 of barrel 11, and a defined cylindrical length, force exerted by plunger 20 increases the pressure of fluid in inner chamber 15 of barrel 11. Additionally, if desired, an additional flow restriction member or orifice can be employed to create a fluid flow restriction to create a beneficial effect on system fluid pressure. Said orifice can be an orifice that is inserted into the body of syringe barrel 11, a feature molded into outlet 13, or attached to the end of syringe outlet 13.

In the illustrative example depicted in FIG. 5, it is to be understood that said plunger 20 has been pushed into said primary inner chamber 15 of barrel 11 faster than fluid can flow from said primary inner chamber 15 through outlet port 13 at less than a predetermined maximum allowable fluid flow rate. For example, if a clinician or other user rapidly pushes plunger 20 into barrel 11, such as when giving a bolus injection, flow rate of fluid displaced through outlet 13—and corresponding fluid pressure in primary inner chamber 15 of barrel 11—can rapidly increase.

If said output flow rate of fluid passing through output 13 exceeds a predetermined maximum value, the corresponding fluid pressure in primary inner chamber 15 of barrel 11 will also exceed a corresponding allowable value. It is to be observed that a corresponding maximum allowable fluid pressure in said primary inner chamber 15 can be determined for a maximum allowable fluid flow rate through outlet 13, either through calculation or empirical observation.

Still referring to FIG. 5, plunger inner piston 23 is no longer seated within seat 25 of plunger piston 21. Said plunger inner piston 23 can be made of elastomer, plastic or other material that beneficially creates a dynamic pressure seal—or at least a tight fit—against the inner surface of plunger inner chamber 22 of plunger 20. A bypass port 26 and plunger seat 25 are formed within plunger piston 21. A pressure control vent 28 can also extend through said plunger 20 into inner chamber 22.

As depicted in FIG. 5, constant force spring 24 applies force to plunger inner piston 23 to bias said plunger inner piston 23 within seat 25 in plunger piston 21. However, said plunger inner piston 21 remains positioned within seat 25 only while fluid pressure in primary inner chamber 15 does not exceed a predetermined acceptable value (which corresponds with a predetermined maximum allowable flow rate through outlet 13). If a user pushes plunger 20 with too much force (generating an output flow rate greater than the maximum allowable flow rate) the fluid pressure in primary inner chamber 15 will increase, thereby creating a greater force acting on plunger inner piston 23 than the opposing force applied by compression spring 24. When said maximum flow rate through outlet 13 is exceeded, plunger inner piston 23 will no longer remain fully seated in seat 25; fluid pressure acting on said plunger inner piston 23 will exceed opposing biasing force applied by compression spring 24, thereby causing said plunger inner piston 23 to become unseated from seat 25 and move within inner chamber 22 of barrel 20.

Plunger inner piston 23 effectively acts as a dynamically movable divider having a first adjustable volume on a first side of said divider, and a second adjustable volume on a second side of said divider. Movement by plunger inner piston 23 away from seat 25 effectively decreases available volume on one side of said plunger inner piston 23, while simultaneously increasing available volume on the other side of said plunger inner piston 23 (which is in communication with fluid in inner chamber 15 of barrel 11). Thus, it is to be observed that the available volume for fluid in primary inner chamber 15 can be automatically and dynamically increased based on the position of plunger inner piston 23 within inner chamber 22. As said plunger inner piston 23 moves away from seat 25, the available volume in fluid communication with inner chamber 15 increases, while the resulting pressure of said fluid (and flow rate through output 13) correspondingly decreases.

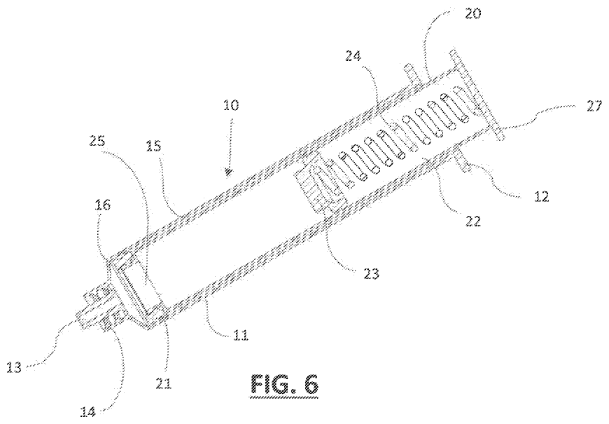

FIG. 6 depicts a side sectional view of a first embodiment output flow regulating apparatus 10 of the present invention in fifth position. In this position, plunger 20 is depicted as being substantially fully inserted or pushed within said inner chamber 15 of barrel, such that plunger piston 21 is proximate to or in contact with distal end 16 of barrel 11. However, plunger inner piston 23 remains unseated from seat 25 of said plunger piston 21. As noted above, plunger inner piston 23 effectively acts as a dynamically movable divider between two separate adjustable volumes. In the position depicted in FIG. 6, the available volume of inner chamber 22 on one side of plunger inner piston 23 has decreased, while the available volume on the opposite side of said plunger inner piston 23 (the side in fluid communication with inner chamber 15 of barrel 11) has increased.

Biasing force remains applied to plunger inner piston 23 by compression spring 24. Thus, when fluid pressure acting on said plunger inner piston 23 has been sufficiently reduced, said biasing spring 24 will push said plunger inner piston 23 toward outlet 13. Thus, in the position depicted in FIG. 6, said biasing force exerted by compression spring 24 on said plunger inner piston 23 will eventually displace any fluid on the leading side of plunger inner piston 23 (that is, the side of plunger inner piston 23 closest to outlet 13) through said outlet 13 at less than or equal to the allowable maximum flow rate.

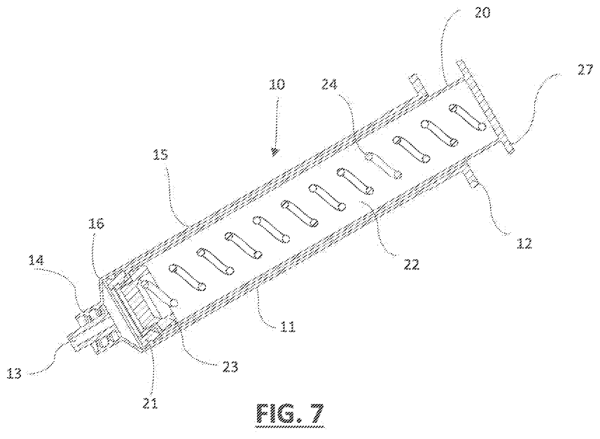

FIG. 7 depicts a side sectional view of a first embodiment output flow regulating apparatus 10 of the present invention. In this position, plunger 20 is depicted as being substantially fully inserted or pushed within said inner chamber 15 of barrel 11, such that plunger piston 21 is proximate to or in contact with distal end 16 of barrel 11. Biasing force applied to plunger inner piston 23 by compression spring 24 has caused fluid within the volume on the leading side of plunger inner piston 23 (that is, the side of plunger inner piston 23 closest to outlet 13) to also be fully displaced by said inner piston 23 through fluid outlet 13. In the position depicted in FIG. 7, plunger inner piston 23 is again seated within plunger piston 21, and output flow regulating apparatus 10 of the present invention is in substantially the same position as depicted in FIG. 2.

In the scenario depicted in FIG. 7, said constant force spring 24 has pushed inner plunger piston 23 at a predetermined force to displace all remaining fluid through outlet 13 at less than the predetermined maximum allowable fluid flow rate. Further, all such remaining fluid is displaced from said output flow regulating apparatus 10 through outlet 13 without the requirement of a secondary or additional procedure or operation.

A user can visually monitor whether the rate of descent of syringe plunger 20 corresponds to a desired output fluid flow rate, versus whether said descent rate of said syringe plunger 20 is too great, depending upon whether inner plunger piston 23 has compressed said spring 24. If secondary inner plunger piston 23 moves from its resting position in plunger seat 25 and has compressed spring 24, a user can selectively apply less force to plunger 20 to slow the descent rate of said syringe plunger 20 (or, when appropriate stop pushing said plunger 20 completely) in order to permit said secondary inner plunger piston 23 to travel back to its resting position.

The present invention regulates output fluid flow rate through outlet 13 so that said flow rate is at or below a predetermined maximum allowable fluid flow rate. Put another way, depending on the rate of descent of plunger 20, fluid will flow from inner chamber 11 of barrel 15 into inner chamber 22 of said plunger 20, or from plunger inner chamber 22 to inner chamber 11 of barrel 15, while also simultaneously flowing out of fluid outlet 13 at or below the maximum designed fluid flow rate. In the embodiment depicted in FIG. 3, when said secondary plunger piston 23 has fully compressed constant force spring 24, plunger inner chamber 22 has sufficient capacity to hold the entire volume of fluid contained within inner chamber 15 of syringe barrel 11.

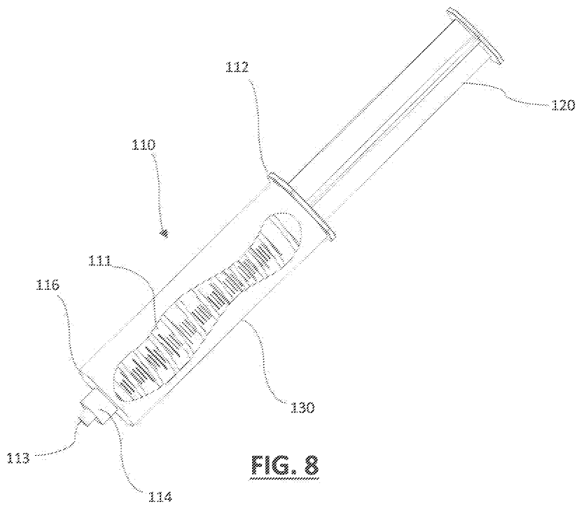

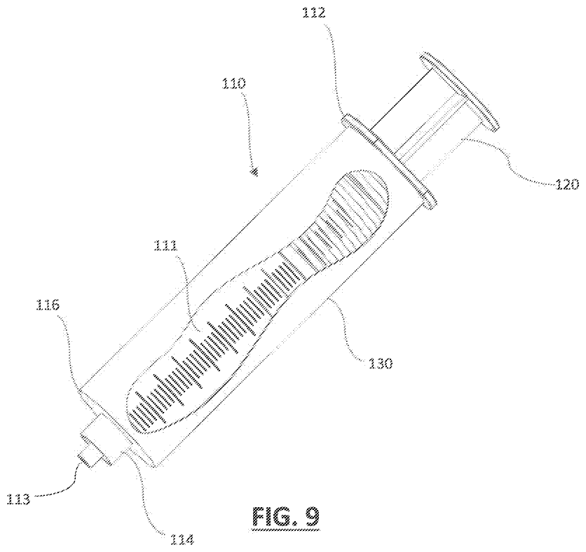

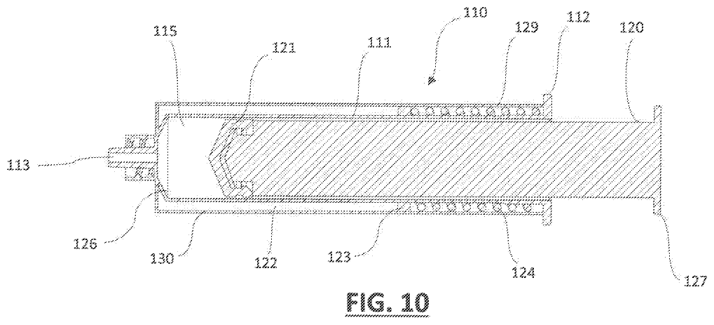

FIG. 8 depicts a perspective and partially cut-away view of a second embodiment output flow regulating apparatus 110 of the present invention in a first position, while FIG. 9 depicts a perspective and partially cut-away view of said second embodiment output flow regulating apparatus 110 of the present invention in a second position. FIG. 10 depicts a side sectional view of second embodiment output flow regulating apparatus 110 of the present invention in a second position. Second embodiment output flow regulating apparatus 110 has substantially the same function as first embodiment 10 discussed above

As depicted in FIGS. 8 and 9, second embodiment output flow regulating apparatus 110 also generally resembles a conventional syringe having a barrel 111 with distal end 116 at one end of said barrel 111, and barrel or finger flanges 112 at the opposite end of barrel 111. Said barrel 111 further comprises fluid outlet 113 and connection assembly 114. A housing member 130 is generally disposed around said barrel 111. Although other connection assemblies can be optionally employed, in a preferred embodiment said connection assembly 114 comprises a Luer lock connection that is well known to those having skill in the art. Said connection assembly 114 can permit operational attachment of said barrel 111 (and fluid outlet 113) to another device or component such as, for example, an output conduit or hypodermic needle. Second embodiment output flow regulating apparatus 110 further comprises a movable plunger 120 that is slidably received within an inner chamber of barrel 111.

Referring to FIG. 10, plunger 120 further comprises plunger piston 121 at one end, and plunger or thumb flange 127 at its opposite end. Plunger piston 121 can be beneficially constructed of an elastomer, plastic or other material that can provide a dynamic fluid seal—or at least fit tightly—against the inner surface of barrel 111. It is to be observed that barrel 111 can be virtually any size, while inner chamber 115 of said barrel 111 can be dimensioned to accommodate virtually any desired volume of liquid. As depicted in FIG. 10, plunger 120 is depicted as being partially withdrawn or pulled from inner chamber 115 of barrel 111; it is to be observed that as plunger 120 is pushed further into barrel 111, the volume of inner chamber 115 is effectively reduced between plunger piston 121 and outlet 113, thereby causing liquid or other fluid to be displaced from said inner chamber 115 through said outlet 113.

Referring to FIG. 10, a supplemental or secondary inner chamber 122 is formed by an annular space defined between barrel 111 and outer housing 130. Ring-like annular piston 123 is movably disposed within said annular inner chamber 122, while force is applied to annular piston 123 to bias said annular piston 123 in a direction away from plunger flange 127 and toward outlet 113. In a preferred embodiment, said biasing force can be applied by compression spring 124; said compression spring 124 can be a constant force coil spring, with barrel 111 disposed through said coil spring. The volume of annular supplemental inner chamber 122 should beneficially be substantially the same or larger than the volume of inner chamber 115 of barrel 111.

Annular inner piston 123 effectively acts as a dynamically movable divider having a first adjustable volume on a first side of said divider, and a second adjustable volume on a second side of said divider. Said first and second volumes can effectively increase or decrease depending upon the position of said annular inner piston 123. As such, said available volume of said annular inner chamber 122 in fluid communication with inner chamber 115 of barrel 111 can be automatically and dynamically adjusted based upon the position of said annular inner piston 123. Supplemental annular inner chamber 122 is in fluid communication with inner chamber 115 of barrel 111 via bypass port 126. In a preferred embodiment, at least one pressure control vent 129 extends through plunger 120 into annular supplemental inner chamber 122. Annular piston 123 can be made of elastomer, plastic or other material that beneficially creates a dynamic pressure seal—or at least a tight fit—against the outer surface of barrel 111 and the inner surface of housing 130.

Because outlet 113 has a relatively small diameter relative to inner chamber 115 of barrel 111, and a defined cylindrical length, said outlet 113 comprises a flow restriction. Force exerted by plunger 120 increases the pressure of fluid in inner chamber 115 of barrel 111. If desired, an orifice can be employed to create an additional fluid flow restriction to create a beneficial effect on system fluid pressure. Said orifice can be an orifice that is inserted into the body of syringe barrel 111, a feature molded into outlet 113, or it can be attached to the end of syringe outlet 113.

In order to permit fluid pressure in inner chamber of 115 of barrel 111 to reach (but not exceed) a desired maximum—and allow a predetermined allowable fluid flow rate through outlet 113—constant force spring 124 applies force to annular inner piston 123 to block bypass port 126. However, said bypass port only remains blocked by annular inner piston 123 when said fluid pressure in inner chamber 115 of barrel 111 is less than a predetermined value for a predetermined maximum allowable fluid flow rate through outlet 113. Thus, if a clinician or other user rapidly pushes plunger 120 into barrel 111 (such as, for example when giving a bolus injection) fluid pressure in inner chamber of 115 of barrel 111 can increase; said fluid pressure is communicated through bypass port 126 and acts on annular inner piston 123.

Eventually, if a maximum allowable fluid pressure in chamber 115 of barrel 111 is exceeded, said fluid pressure will impart greater force on annular inner piston 123 than the force applied by compression spring 124, thereby causing annular inner piston 123 to move within annular supplemental inner chamber 122 generally in a direction away from outlet 113. Thus, annular inner piston 123 effectively acts as a dynamically movable divider between two separate adjustable volumes. A first available volume of inner chamber 122 on one side of annular inner piston 123 can decrease, while a second available volume on the opposite side of said annular inner piston 123 can increase.

Biasing force remains applied to annular inner piston 123 by compression spring 124 and biases said annular inner piston 123 toward outlet 113. Thus, when opposing fluid pressure acting on said annular inner piston 123 falls below a certain predetermined value, said biasing force from compression spring 124 causes said annular inner piston 123 to generally move in the direction toward outlet 113 and displace any fluid on the leading side of said annular inner piston 123 (that is, the side of annular inner piston 123 closest to outlet 113) through said outlet 113 at less than or equal to the allowable maximum flow rate.

A user of second embodiment output flow regulating apparatus 110 can also visually monitor whether the rate of descent of syringe plunger 120 corresponds to a desired output fluid flow rate (versus whether said descent rate of said syringe plunger 120 is too great) depending upon whether annular inner piston 123 has compressed said compression spring 124. If annular inner piston 123 moves from its resting position near distal end 116 of barrel 111 and has compressed spring 124, a user can selectively apply less force to plunger 120 in order to slow the descent rate of said syringe plunger 120, or stop pushing said plunger 120 completely, in order to permit said annular inner piston 123 to travel back to its resting position.

The present invention regulates output fluid flow rate through outlet 113 so that said flow rate remains at or below—and does not exceed—a predetermined maximum allowable fluid output flow rate through outlet 113. Depending on the descent rate of plunger 120, fluid will flow from inner chamber 111 of barrel 115 into annular supplemental inner chamber 122, or from said annular supplemental inner chamber 122 to inner chamber 111 of barrel 115, while also simultaneously flowing out of fluid outlet 113 at or below said maximum predetermined fluid flow rate. In the embodiment depicted in FIG. 10, when said annular inner piston 123 has fully compressed constant force spring 124, annular supplemental inner chamber 122 should beneficially have sufficient volumetric capacity to hold the entire volume of fluid within inner chamber 115 of syringe barrel 111.

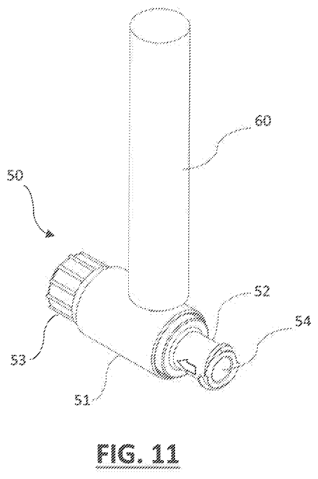

FIG. 11 depicts a side perspective view of a third embodiment output flow regulating apparatus 50 of the present invention. In a preferred embodiment, said third embodiment output flow regulating apparatus 50 generally comprises body member 51, first connection member 52, second connection member 53 and through bore 54. Said third embodiment output flow regulating apparatus 50 further comprises supplemental container 60.

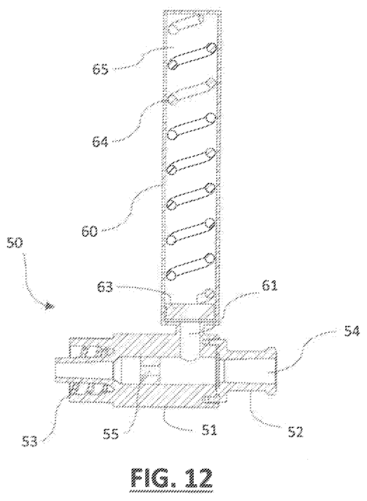

FIG. 12 depicts a side sectional view of said third embodiment output flow regulating apparatus 50 of the present invention. Said third embodiment output flow regulating apparatus 50 generally comprises body member 51, first connection member 52, second connection member 53 and through bore 54. An optional inner flow orifice 55 can be disposed within said through bore 54; said optional flow orifice 55 can comprise a fluid flow restriction having predetermined dimensions. Internal flow port 61 extends from said through bore 54 into inner chamber 65 of supplemental container 60. Inner piston 63 is movably disposed within said inner chamber 65 of supplemental container 60 and can be constructed of elastomer, plastic or other material that beneficially creates a dynamic pressure seal—or at least a tight fit—against the inner surface of said inner chamber 65. Compression spring 64 biases said inner piston 63 toward internal flow port 61.

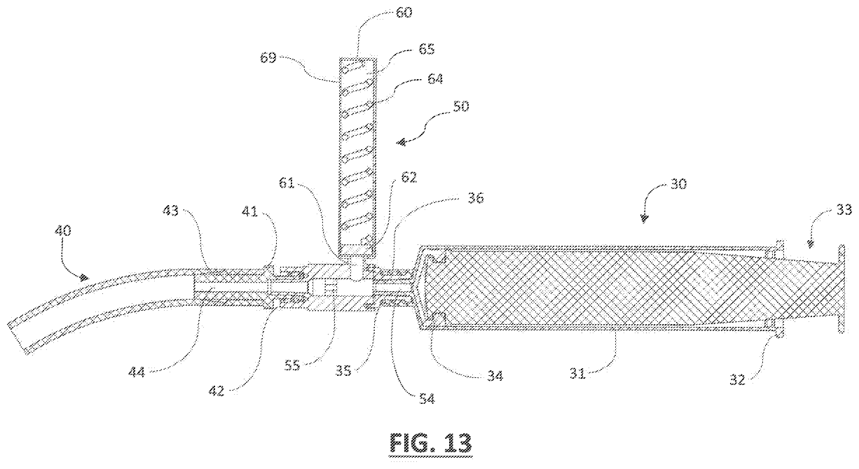

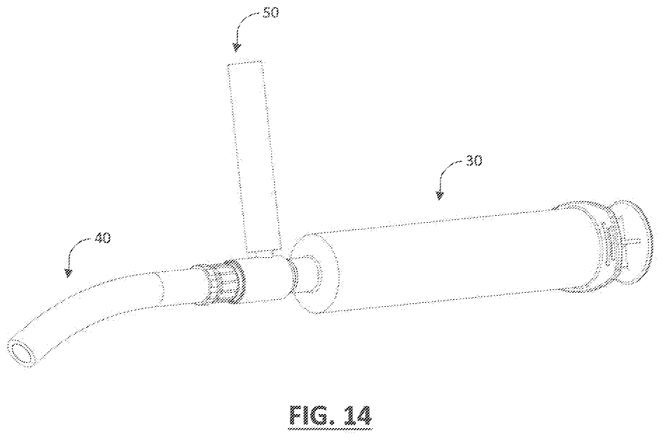

FIG. 13 depicts a side sectional view of third embodiment output flow regulating apparatus 50 of the present invention connected between a conventional syringe 30 and fluid conduit 40. Although designs may vary depending upon different factors, as depicted in FIG. 13 conventional syringe 30 generally comprises barrel 31 having barrel/finger flanges 32, outlet 35 and connection member 36. Plunger 33 has plunger piston 34 and is movably disposed within barrel 31. Still referring to FIG. 13, fluid conduit 40 can comprise flexible plastic tubing, such as is commonly used and well known in the medical field for administering medications. Connector 41 has connection member 42, extension 44 and external barb 43. Output flow regulating apparatus 50 of the present invention is interposed between conventional syringe 30 and fluid conduit 40 and is in fluid communication with both said conventional syringe 30 and said fluid conduit 40. FIG. 14 depicts a side perspective view of a third embodiment output flow regulating apparatus 50 of the present invention installed on conventional syringe 30 with flow conduit 40.

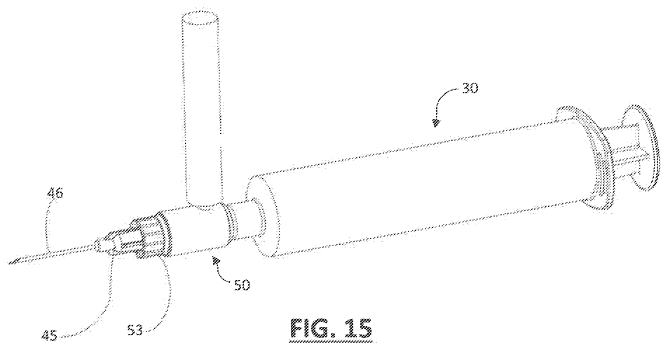

FIG. 15 depicts a side perspective view of an alternative configuration for third embodiment output flow regulating apparatus 50 of the present invention. Instead of flow conduit 40 depicted in FIGS. 13 and 14, a conventional hub 45 is connected to connection member 53. Hub 45 is connected to conventional hypodermic needle 46. Output flow regulating apparatus 50 of the present invention is interposed between conventional syringe 30 and hypodermic needle 46 and is in fluid communication with both said conventional syringe 30 and said hypodermic needle 46.

Output flow regulating apparatus 50 of the present invention can be selectively attached to the fluid output of a fluid dispensing device (such as, for example, conventional syringe 30) in order to beneficially control the output flow rate from said fluid dispensing device. In a preferred embodiment, a known fluid back pressure is generated between the inlet and outlet of said output flow regulating apparatus 50; by way of illustration, but not limitation, said fluid backpressure can be created by include a fluid flow restriction—such as inner orifice 55 depicted in FIG. 12—which has a reduced flow port diameter.

Referring back to FIG. 13, as fluid flows from the outlet of a dispensing apparatus (such as conventional syringe 30) into through bore 54 of output flow regulating apparatus 50, fluid backpressure is generated when said fluid flows through the restriction formed by inner orifice 55. A flow rate for said fluid can be determined (using Poiseuille's Law and other fluid dynamics principles, for example). Inner piston 62 is movably disposed within inner chamber 65 of supplemental container 60; biasing force is applied to inner piston 62 by compression spring 64. A vent 69 extends through supplemental container 60.

Constant force compression spring 64 applies sufficient biasing force to inner piston 62 to cause said inner piston 62 to block internal flow port 61 only until opposing fluid pressure acting on said inner piston 62 exceeds a predetermined desired value (corresponding to said maximum allowable flow rate). When fluid backpressure created by flow restriction of inner orifice 55 reaches a certain predetermined value (corresponding to a maximum allowable fluid flow rate through said orifice 55), inner piston 62 can move within inner chamber 65 of supplemental container 60 and unblock flow port 61. Put another way, if a user pushes plunger 33 of conventional syringe 30 with too much force (that is, a force that causes fluid flow through inner orifice 55 to exceed a predetermined maximum value), the fluid pressure upstream of inner orifice 55 will increase, thereby imparting a greater force on inner piston 62 than the opposing force applied by compression spring 64. When this occurs, inner piston 62—which acts as divider between first and second adjustable volumes—moves within inner chamber 65 of supplemental container 60, thereby increasing the available volume for fluid upstream of inner orifice 55. By dynamically adjusting said available volume for fluid upstream of inner orifice 55, fluid pressure and corresponding flow rate through said inner orifice 55 are reduced.

The present invention regulates output fluid flow rate through inner orifice 55 (as well as downstream components, such as a fluid conduit or hypodermic needle) so that said fluid flow rate remains at or below a predetermined maximum allowable fluid flow rate. If a clinician or other user rapidly pushes plunger 33 of conventional syringe 30, (such as, for example, when giving a bolus injection) fluid pressure will act on inner piston 62 with a force that opposes the force exerted by constant force compression spring 64. If said inner piston 62 has fully compressed constant force compression spring 64, the available volume on the side of piston 62 facing flow port 61 should beneficially be sufficient to hold the entire volume of fluid contained within barrel 31 of said conventional syringe 30.

In a preferred embodiment, a user can select a flow control apparatus 50 having a desired maximum fluid flow rate capacity. For example, in a medical setting, a user can determine said desired flow rate capacity based on a particular drug being administered to a patient. Using a conventional syringe of a desired size and with a compatible connection, said user can draw the prescribed or predetermined volume of fluid (e.g., medicine) into said syringe. Output flow control apparatus 50 can be selected from multiple selections having desired specifications and can be attached to the outlet of said syringe and an inlet of a fluid conduit (such as a tube). In this configuration, output flow regulating apparatus 50 of the present invention is beneficially interjected in a flow path between said syringe and a patient, thereby beneficially regulating or controlling the maximum allowable flowrate of fluid discharged from said syringe.

It is to be observed that desired output fluid flow rate can vary depending on a number of different factors; such factors include, but are not necessarily limited to, medication concentration, patient size, patient health and/or many other variables. Health care personnel can benefit from an adjustable apparatus that can be used to achieve various different flow rates with a single device. In order to allow a user to adjust fluid output flow rate, certain features of the present invention can affect internal fluid pressures which, in turn, can effect fluid flow rate. These features include the constant force spring, the surface area of the secondary plunger(s) and the orifice size in the outlet.

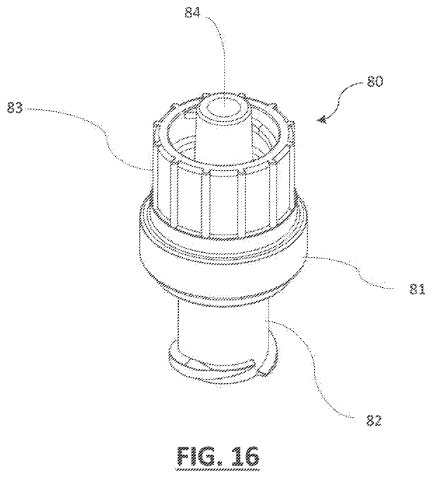

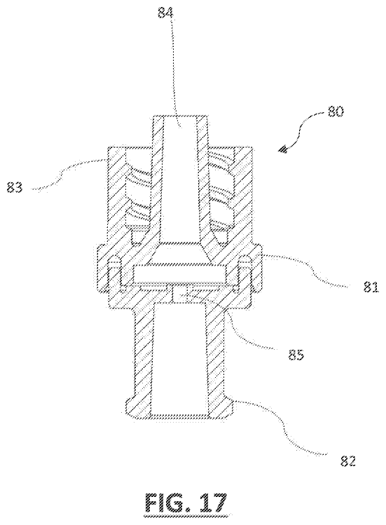

FIG. 16 depicts an isometric view of an orifice attachment 80 of the present invention, while FIG. 17 depicts a sectional view of said orifice attachment 80. As discussed herein, it can be beneficial to include an orifice or other flow restriction in a fluid flow stream and can increase flowing pressure of said fluid for purposes of the present invention. Further, in certain circumstances, it may be beneficial to permit selective adjustment of the dimensions of such an orifice or other flow restriction.

Referring to FIG. 17, orifice attachment 80 generally comprises body member 81, inlet connection member 82, outlet connection member 83 and through bore 84. Referring to FIG. 17, orifice attachment 80 generally comprises body member 81, inlet connection member 82 and outlet connection member 83, while through bore 84 generally extends through said attachment member 80 from said inlet connection member 82 to said outlet connection member 83. Internal orifice or flow restriction 85 having desired dimensions is disposed at a desired location withing the flow channel formed by said through bore 84.

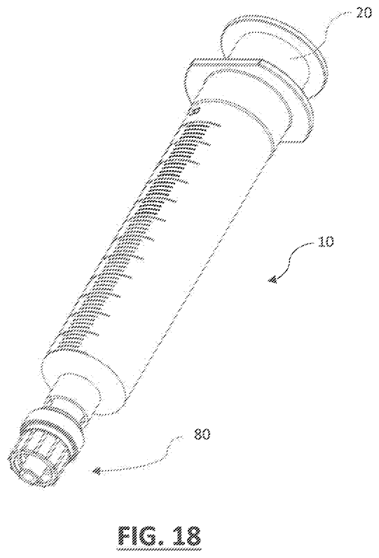

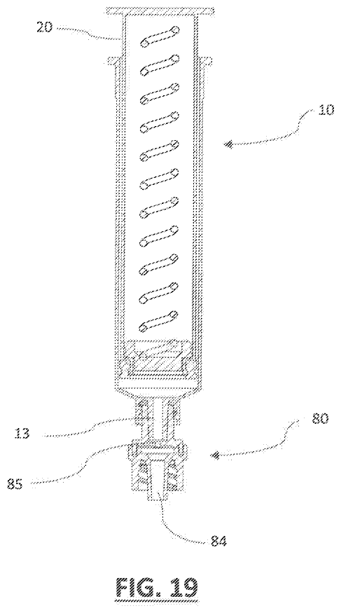

FIG. 18 depicts a side perspective view of orifice attachment 80 of the present invention installed on a syringe, such as a first embodiment output flow regulating apparatus 10 of the present invention. FIG. 19 depicts a side sectional view of said orifice attachment 80 of the present invention installed on said output flow regulating apparatus 10. Orifice attachment 80 generally comprises an interchangeable apparatus that can be quickly and easily connected to the fluid outlet of a dispensing device (such as a syringe) in order to interpose an orifice or flow restriction having desired dimensions into a fluid flow stream.

Referring to FIG. 19, orifice attachment 80 of the present invention can be operationally attached to outlet 13 of output flow regulating apparatus 10 via slot 18. Pressurized fluid output from outlet 13 flows into through bore 84 of said orifice attachment 80 and through internal orifice 85. It is to be observed that the dimensions of an orifice or flow restriction (such as internal orifice 85) can be selectively changed or adjusted simply by disconnecting orifice attachment 80 from outlet 13 of output flow regulating apparatus 10. Thereafter, a different or alternative orifice attachment 80—which can have an internal orifice having different desired dimensions—can then be installed on said output flow regulating apparatus 10. In this manner, different orifice attachments 80 having desired orifice dimensions can be utilized to selectively restrict fluid flow and generate a desired backpressure in said fluid.

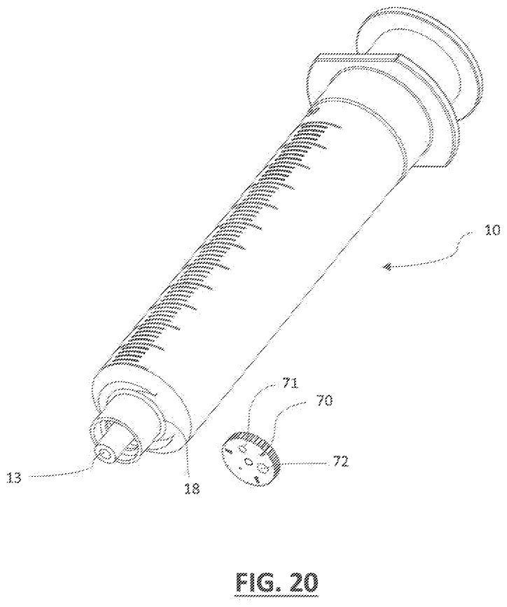

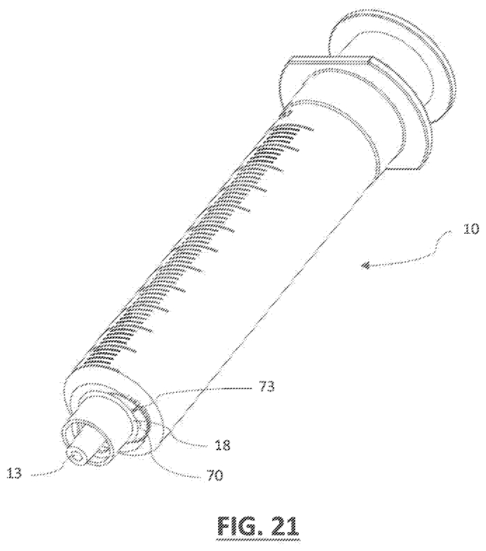

FIG. 20 depicts a side perspective and partially exploded view of a modified output flow regulating apparatus 10 having slot 18 and adjustable orifice disk 70, while FIG. 21 depicts a side perspective view of said modified output flow regulating apparatus 10 with said orifice disk 70 installed in slot 18 of said modified output flow regulating apparatus 10. In a preferred embodiment, said orifice disk 70 has a plurality of orifices 71 and 72, which can beneficially be of different varying sizes and/or geometries. Said orifice disk 70 can also have rough outer edge 73, which can help a user grasp or rotate said orifice disk 70 using fingers. Orifice disk 70 can be selectively rotated or otherwise re-positioned within slot 18 in order to position a desired orifice (such as alternative orifices 71 or 72) in a fluid flow stream. As such, orifice disk 70 allows a user to selectively adjust flow restriction—and thus fluid flow rate—through outlet 13 of output flow regulating apparatus 10.

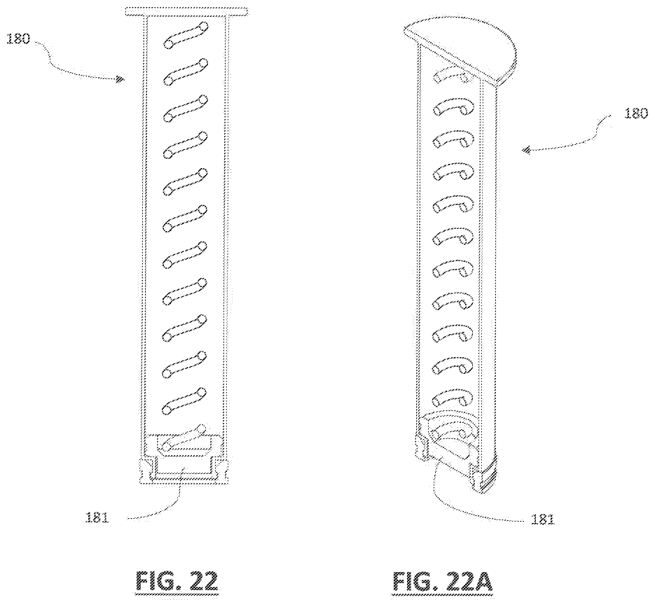

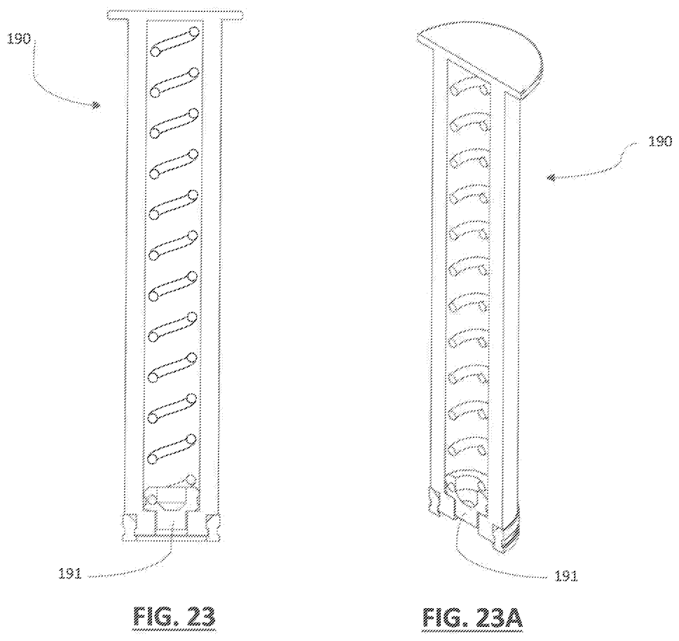

FIG. 22 depicts a side sectional view of a first alternative plunger 180 of the present invention, while FIG. 22A depicts a perspective sectional view of said first alternative plunger 180 of the present invention. FIG. 23 depicts a side sectional view of a second alternative plunger 190, while FIG. 23A depicts a perspective sectional view of said second alternative plunger 190.

Referring to FIGS. 22, 22A, 23 and 23A, it is to be observed that by changing the surface area of plunger piston 181 and 191, the amount of force acting on said plunger piston can be adjusted or varied for a given fluid pressure. As such, plunger 20 depicted in FIGS. 18 and 19 can be removed from barrel 11 and selectively replaced with first alternative plunger 180 (having a plunger piston 181 having a larger surface area than plunger piston of plunger 20), or second alternative plunger 190 (having a plunger piston 191 having a smaller surface area than plunger piston of plunger 20).

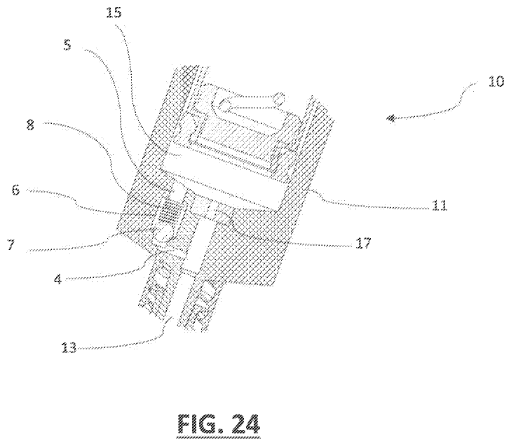

FIG. 24 depicts a detailed sectional view of a first embodiment output flow regulating apparatus 10 of the present invention further comprising an internal flow orifice 17 and a check valve assembly. It is to be observed that presence of internal orifice 17 may make it difficult or time consuming to pull or draw fluid (such as from a vial or other medication storage container) into inner chamber 15 of barrel 11. As such, a check valve assembly will allow unrestricted fluid flow into inner chamber 15 of barrel 11 but will not negatively impact fluid flow rate when medication or other fluid is displaced from said inner chamber 15 through orifice 17 and outlet 13.

Referring to FIG. 24, said check valve assembly generally comprises bypass channels 4 and 5, and valve chamber 6 disposed between said bypass channels 4 and 5. Ball 7 and compression spring 8 are disposed within said chamber 6; compression spring 8 is configured to bias ball 7 against an opening of bypass channel 4. When fluid is being drawn into inner chamber 15 of barrel 11 fluid pressure acts on said ball 7 and compresses spring 8, thereby allowing ball 7 to move away from the opening of bypass channel 4. Said fluid flows into valve chamber 6, around ball 7, through bypass channel 5, and into inner chamber 15 of barrel 11. Conversely, when plunger 20 is depressed and fluid is displaced out of said inner chamber 15 of barrel 11, fluid pressure (and force from compression spring 8) act on ball 7, forcing it against the opening to bypass channel 4 and obstructing/blocking fluid flow through said bypass channel. As such, said check valve assembly permits fluid flow into inner chamber 15 of barrel 11 through bypass channel 4, valve chamber 6 and bypass channel 5, yet does not permit fluid flow through said components in the opposite direction.

As discussed in detail herein, referring to FIGS. 2 through 7, constant force spring 24 in plunger inner chamber 22 applies force to inner plunger piston 23 which, in turn, impacts system fluid pressure and output fluid flow rate of the present invention. In one embodiment, thumb flange 27 can be attached to a removable cap. Said removeable cap can be selectively removed to permit access to constant force spring 24. Said constant force spring 24 can be removed and replaced with an alternative constant force spring having different properties (typically biasing force that is applied to inner piston plunger 23).

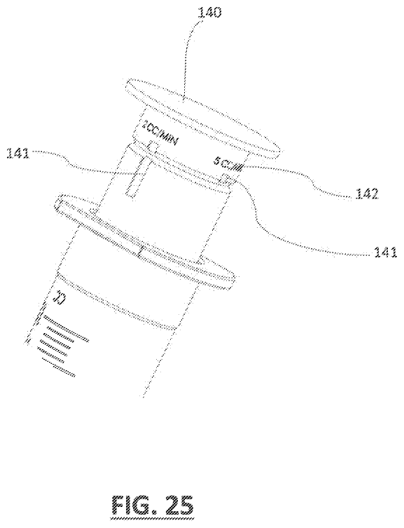

FIG. 25 depicts a detailed perspective view of an adjustable-rate cap assembly of the present invention having cap 140. Cap 140 can include reference marker 141 and indicia 142 (such as flow rate). Cap 140 which is in contact with one end compression spring 24 (not visible in FIG. 25) can have a tapered surface. Cap 140 can be selectively twisted to different positions, thereby resulting in the tapered surface under cap 140 exerting greater or lesser force on said compression spring which, in turn, imparts greater or lesser force on an plunger inner piston. Adjusting said force imparted by said compression spring allows a user to change the regulated output fluid flow rate of the apparatus.

An alternative embodiment of the present invention permits measurement, storage and/or transmission of flow rate data from a fluid dispensing apparatus such as a syringe. Such information regarding fluid flow rate can be extremely important, particularly flow rates of critical care drugs being administered. Conventional flow meters for this purpose are notoriously inaccurate, large and expensive. As discussed herein, fluid dynamics principles permit a means for correlating pressure differential in a system to a flow rate. In a preferred embodiment, the present invention can measure fluid pressure in at least two places—namely, both upstream and downstream of a flow restriction or orifice having known dimensions.

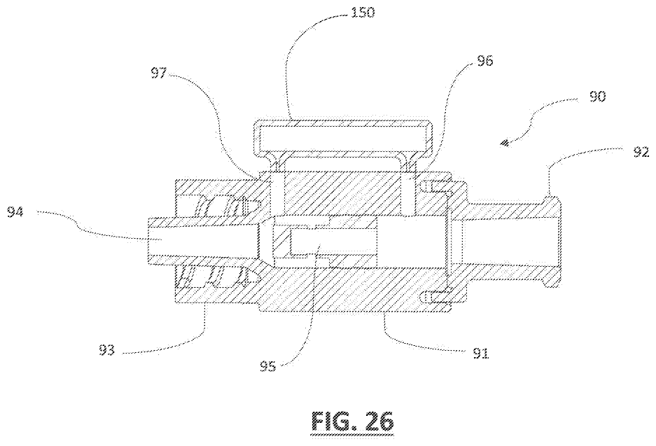

FIG. 26 depicts a side sectional view of an output flow measurement apparatus 90 of the present invention. Said output flow measurement apparatus 90 generally comprises body member 91, first connection member 92, second connection member 93 and through bore 94. Inner flow orifice 95 can be disposed within said through bore 94; said flow orifice 95 can comprise a fluid flow restriction having predetermined or desired dimensions. Upstream measurement port 96 and downstream measurement port 97 both extend through said body member 91; upstream measurement port 96 is positioned on the upstream (inlet) side of flow orifice 95, while downstream measurement port 97 is positioned on the downstream (outlet) side of said flow orifice 95. Controller module 150 can be mounted to an external surface of output flow measurement apparatus 90.

Output flow measurement apparatus 90 measures fluid pressures both upstream and downstream of flow orifice 95, and uses such information to determine fluid flow rate of output from said output flow apparatus 90. Data or information such as fluid flow rate, cumulative volume injected, and injection pressure can be transmitted via wireless connection to a receiving station (such as a hospital monitor). Said data can also be recorded and/or displayed. Alternatively, said data can be stored locally on said output flow measurement apparatus 90 to be extracted with the use of a conventional hospital scanner, or wired or wireless download mechanism. Said data can also be added to a patient chart, used in connection with a malpractice claim, or billing/monetization of procedure(s).

Output flow measurement apparatus 90 can beneficially include an alarm to sound an audible signal and/or have a visual indication such as a light or readout when manual regulation of administration rate is desired. The present invention senses fluid pressure and determines flow rate; if said flow rate exceeds a desired flow rate, an audible alarm and/or a visual signal notifies a user that said flow rate has been exceeded. Thus, when recognizing such an alarm, said user can slow the descent speed of a plunger to reduce the output flow rate to an acceptable flow rate. In addition, data recordings can be used to corroborate clinician's actions and compliance with applicable requirements or guidelines.

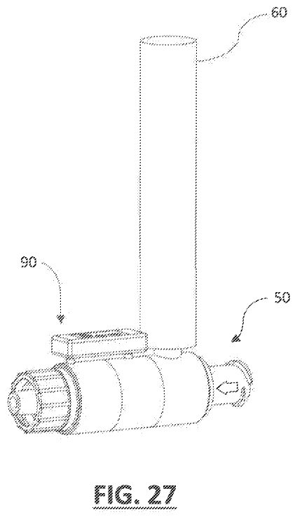

FIG. 27 depicts a side perspective view of output flow measurement apparatus 90 of the present invention equipped with output flow regulating apparatus 50 having a supplemental fluid container 60. It is to be observed that said output flow regulating apparatus 50 having a supplemental fluid container 60 can regulate fluid flow as described more fully herein so that fluid flow rate through said apparatus does not exceed a predetermined maximum rate. Additionally, output flow measurement apparatus 90 can measure, display and/or record said flow rate in order to confirm that said fluid flow rate is less than or equal to said predetermined maximum flow rate.

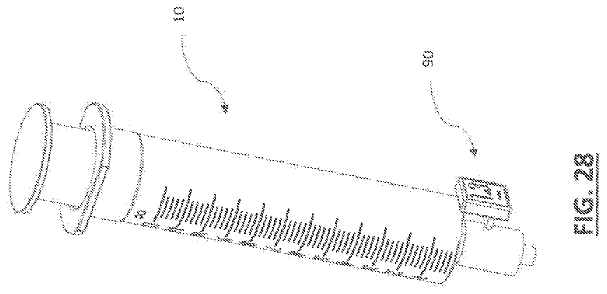

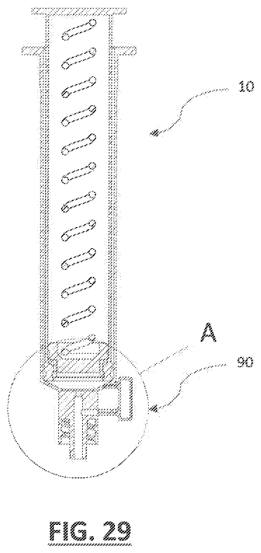

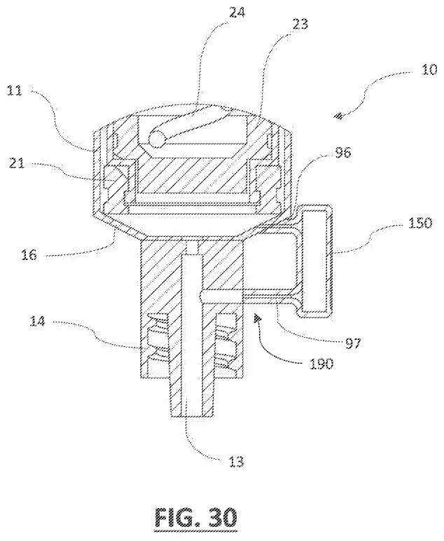

Similarly, FIG. 28 depicts a side perspective view of an output flow measurement apparatus 90 of the present invention installed on an output flow regulating apparatus 10 of the present invention. FIG. 29 depicts a side sectional view of output flow measurement apparatus 90 equipped with output flow regulating apparatus 10 of the present invention, while FIG. 30 depicts a detailed view of Detail “A” of FIG. 29. It is to be observed that said output flow regulating apparatus 10 can regulate fluid flow so that flow rate through said apparatus does not exceed a predetermined maximum rate as discussed in detail herein. As with the embodiment depicted in FIG. 27, output flow measurement apparatus 90 can measure, display and/or record said flow rate in order to confirm that said measured flow rate is less than or equal to said predetermined maximum flow rate. For example, information that can be displayed include, without limitation flow rate, injection duration and programmed desired flow rate.

Some embodiments of the present invention may be implemented in one or a combination of hardware, firmware, and software. Hardware may include a hardware processor and memory. The processor may be a microprocessor, central processing unit (CPU), or other types of circuitry. The memory may include volatile memory and non-volatile memory, and other types of memory. The memory may store code (e.g., instructions, logic and/or commands) executed by the processor in the control of the present invention. In some examples, the processor and memory may be collectively referred to as a controller or computing system. The computing system may include an integrated circuit, a printed circuit board (PCB), a printed circuit assembly (PCA) or printed circuit board assembly (PCBA), an application-specific integrated circuit (ASIC), a programmable logic controller (PLC), a component of a distributed control system (DCS), a field-programmable gate array (FPGA), or other types of circuitry.

Some embodiments may also be implemented as instructions stored on a machine-readable medium, which may be read and executed by a computing platform to perform certain operations. A machine-readable medium may include any mechanism for storing or transmitting information in a form readable by a machine, e.g., a computer. For example, a machine-readable medium may include read only memory (ROM); random access memory (RAM); magnetic disk storage media; optical storage media; flash memory devices; or electrical, optical, acoustical or other form of propagated signals, e.g., carrier waves, infrared signals, digital signals, or the interfaces that transmit and/or receive signals, among others.

Firmware may be employed. In some cases, firmware if employed may be code embedded on a controller such as programmed into, for example, ROM or flash memory. Firmware may be instructions or logic for the controller hardware and may facilitate control, monitoring, data manipulation, and so on, by the controller. Remote computing systems may include communicative couplings or circuitry to facilitate computer-implemented control of measurement devices and/or transmission of data.

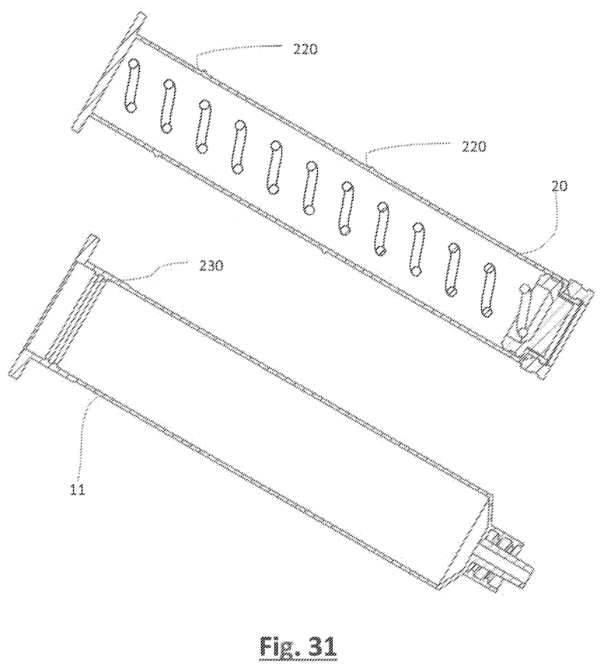

FIG. 31 depicts a side sectional and partially exploded view of a plunger stop mechanism of the present invention. Plunger 20 includes at least one protrusion 220 extending radially outward from the outer surface of said plunger body. Similarly, at least one channel 230 is disposed within and extends around the inner surface of barrel 11; said at least one channel 230 is oriented substantially perpendicular to the longitudinal axes of plunger 20 and barrel 11. In a preferred embodiment, said at least one protrusion 220 is dimensioned to be received within said channel 230 and “lock” or secure said plunger 20 against movement along its longitudinal axis. Notwithstanding the foregoing, it is to be observed that, alternatively, said protrusion can protrude radially inward from said inner surface of said barrel, and said channel can be formed on the outer surface of said plunger body.

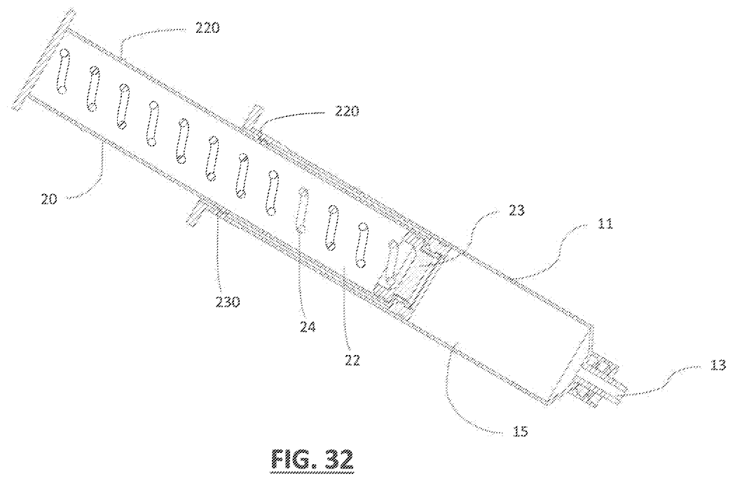

FIG. 32 depicts a side sectional view of a plunger stop mechanism of the present invention. Plunger 20 is depicted as being at least partially pushed into inner chamber 15 of barrel 11 and includes at least one protrusion 220 extending radially outward from the outer surface of said plunger body. At least one channel 230 is disposed within and extends around the inner surface of barrel 11 and said at least one channel 230 is oriented substantially perpendicular to the longitudinal axes of plunger 20 and barrel 11.

As discussed above, if plunger 20 is inserted or pushed within said inner chamber 15 of barrel 11 too quickly (i.e., wherein the maximum discharge flow rate of fluid from chamber 15 flowing through outlet 13 would be exceeded), fluid pressure acting on said plunger inner piston 23 exceeds biasing force applied by compression spring 24, thereby causing said fluid to unseat said plunger inner piston 23. Eventually, biasing force applied to inner piston 23 by compression spring 24 can overcome said fluid pressure, causing plunger inner piston 23 to displace fluid through fluid outlet 13. In this scenario, hydraulic forces act on said plunger 20, thereby causing said plunger 20 to move along its longitudinal axis and withdraw from said barrel 11. However, when said at least one protrusion 220 of plunger 20 is received within said at least one channel 230 disposed on the inner surface of barrel 11, plunger 20 is secured or “locked” against such movement relative to barrel 11.

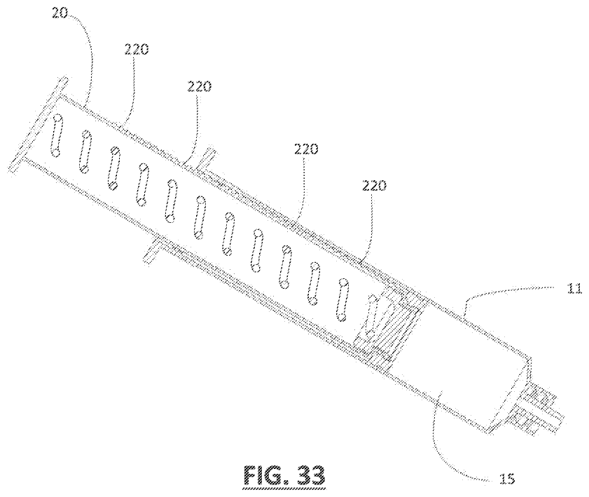

FIG. 33 depicts a side sectional view of an alternative plunger stop mechanism of the present invention. As depicted in FIG. 33, plunger 20 has a plurality of protrusions 220 that extend radially outward from said plunger 20; said protrusions 220 can be arranged in linear alignment and spaced relationship. As such, plunger 20 can be selectively secured or “locked” against movement relative to barrel 11 in multiple different desired positions or locations as plunger 20 is pushed into chamber 15 of barrel 11.

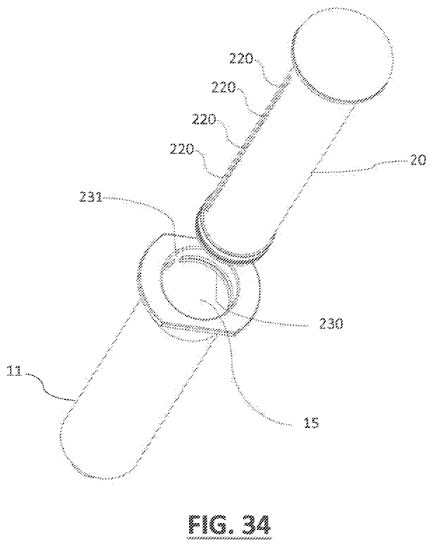

FIG. 34 depicts a perspective and partially exploded view of a said alternative plunger stop mechanism of the present invention. At least one channel 230 is disposed within and extends partially around the inner surface of barrel 11; however, at least one gap or break 231 is formed within said channel 230. As such, when this locking feature is not desired for any reason, plunger 20 can be selectively rotated about its longitudinal axis so that protrusions 220 are aligned with said gap or break 231. In this configuration, plunger 20 can be pushed into, or pulled out of, chamber 15 of barrel 11 without being secured or “locked” against movement relative to barrel 11.

The above-described invention has a number of particular features that should preferably be employed in combination, although each is useful separately without departure from the scope of the invention. While the preferred embodiments of the present invention are shown and described herein, it will be understood that the invention may be embodied otherwise than herein specifically illustrated or described, and that certain changes in form and arrangement of parts and the specific manner of practicing the invention may be made within the underlying idea or principles of the invention.

What is claimed:

1. An apparatus for regulating fluid flow rate from a fluid dispensing device comprising:
a) a primary fluid chamber;
b) an outlet in fluid communication with said primary fluid chamber;
c) a pump member for displacing fluid from said primary fluid chamber through said outlet;
d) a secondary chamber;
e) a dividing member movably disposed in said secondary chamber;
f) a first adjustable volume on a first side of said dividing member, wherein said first volume is in fluid communication with said primary fluid chamber; and
g) a second adjustable volume on a second side of said dividing member; wherein said dividing member moves to increase said first adjustable volume when fluid displaced through said outlet exceeds a predetermined flow rate.

2. The apparatus of claim 1, further comprising a biasing member for resisting movement of said dividing member.

3. The apparatus of claim 2, wherein said biasing member comprises a constant force compression spring.

4. The apparatus of claim 1, wherein said fluid dispensing device comprises a syringe.

5. The apparatus of claim 1, further comprising a flow restriction disposed between said primary chamber and said outlet.

6. A fluid flow rate regulating syringe comprising:
a) a barrel defining an inner chamber;
b) an outlet in fluid communication with said inner chamber of said barrel;
c) a plunger movably disposed in said barrel for displacing fluid through said outlet;
d) a secondary chamber;
e) a dividing member movably disposed in said secondary chamber;
f) a first adjustable volume on a first side of said dividing member, wherein said first adjustable volume is in fluid communication with said inner chamber of said barrel; and
g) a second adjustable volume on a second side of said dividing member; wherein said dividing member moves to increase said first adjustable volume when fluid displaced through said outlet exceeds a predetermined flow rate.

7. The fluid regulating syringe of claim 6, further comprising a biasing member for resisting movement of said dividing member.

8. The fluid regulating syringe of claim 7, wherein said biasing member comprises a constant force compression spring.

9. The fluid regulating syringe of claim 6, further comprising a flow restriction disposed between said inner chamber of said barrel and said outlet.

10. The fluid regulating syringe of claim 6, wherein said secondary chamber is disposed within said plunger.

11. The fluid regulating syringe of claim 6, further comprising an outer housing disposed around at least a portion of said barrel, wherein said secondary chamber comprises an annular space formed between said outer housing and said barrel.

12. The fluid regulating syringe of claim 7, wherein fluid pressure in said primary chamber acts on said dividing member to overcome force imparted by said biasing member when fluid displaced through said outlet exceeds a predetermined flow rate.

13. The fluid regulating syringe of claim 6, further comprising a check valve assembly configured to allow fluid to bypass said outlet while being drawn into said inner chamber of said barrel.

14. A method for regulating fluid output flow rate from a syringe comprising:
a) providing a fluid regulating syringe comprising:
i) a barrel defining an inner chamber;
ii) an outlet in fluid communication with said inner chamber of said barrel;
iii) a plunger movably disposed in said barrel for displacing fluid through said outlet;
iv) a secondary chamber;
v) a dividing member movably disposed in said secondary chamber, wherein a first adjustable volume in fluid communication with said inner chamber of said barrel is defined on a first side of said dividing member, and a second adjustable volume on a second side of said dividing member;
b) drawing fluid into said barrel;
c) displacing fluid from said barrel through said outlet; and
d) moving said dividing member to increase said first adjustable volume when fluid displaced through said outlet exceeds a predetermined flow rate.

15. The method of claim 14, wherein said fluid regulating syringe further comprises a biasing member for resisting movement of said dividing member.

16. The method of claim 15, wherein said biasing member comprises a constant force compression spring.

17. The method of claim 14, wherein said secondary chamber is disposed within said plunger.

18. The method of claim 14, wherein said fluid regulating syringe further comprises an outer housing disposed around at least a portion of said barrel, and wherein said secondary chamber comprises an annular space formed between said outer housing and said barrel.

19. The method of claim 14, wherein fluid pressure in said primary chamber acts on said dividing member to overcome force imparted by said biasing member when fluid displaced through said outlet exceeds a predetermined flow rate.

20. The method of claim 19, wherein surface area of said dividing member is adjusted to change the amount of fluid pressure required to displace said dividing member.
